(19) United States
(12) Patent Application Publication
(10) Pub. No.: US 2007/0148094 A1
(43) Pub. Date: Jun. 28, 2007

(54) Polymeric imaging agents and medical imaging methods

(76) Inventors: Egidijus Edward Uzgiris, Schenectady, NY (US); Stephen Johnson Lomnes, Philadelphia, PA (US); Anup Sood, Clifton Park, NY (US); Kathleen Bove, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/317,945

(22) Filed: Dec. 22, 2005

(51) Int. Cl. A61K 49/10

(52) U.S. Cl. 424/9.34; 424/9.361; 424/9.6

(57) ABSTRACT

Provided herein are image-enhancing agents that are useful for managing disease by imaging disease tissue using magnetic resonance imaging techniques, optical imaging techniques, and a combination of magnetic resonance imaging techniques and optical imaging techniques. Also provided are methods of imaging disease tissue and managing disease using the image-enhancing agents.

### FIELD OF THE INVENTION

The present invention is directed to the field of medical imaging. In particular, the present invention is directed to image-enhancing agents and medical imaging methods using magnetic resonance imaging techniques and optical imaging techniques.

### BACKGROUND

Management of disease conditions is facilitated by various imaging techniques that permit a physician to distinguish healthy tissue from disease tissue and direct treatment to the disease tissue. In particular, surgical success in the management of cancer depends on a number of prognostic factors including the width of the margin of excision. The ability of surgeons to establish clear margins depends on their ability to visualize the difference between healthy and diseased tissue, which in many cases is challenging. Functionally labeling tumors provides to surgeons a real-time and high-resolution functional image that improves their ability to achieve clear margins of excision.

A number of imaging modalities have been used for tumor imaging each with its own benefits. MR agents provide high resolution and are routinely used non-invasively. MR agents do not have general real-time operative applications because the magnetic properties of the instruments and equipment in operating rooms mask magnetic signals and their application is limited to a few clinical sites specializing in MR intraoperative procedures. Optical imaging is typically limited to shallow tumors because absorbance and diffraction of light reduce resolution in deep tissue applications.

### BRIEF DESCRIPTION

The present invention will be made more apparent from the description, drawings and claims that follow.

Disclosed herein are image-enhancing agents comprising a polyamino acid polymer comprising multiple amino residues; a plurality of magnetic resonance imaging moieties covalently bound to a polyamino acid polymer through an amine group; and at least one optical imaging moiety comprising polymethine cyanine dye covalently bound to a polyamino acid polymer through an amine group, wherein at least 1% of the amino residues of the polyamino acid polymer includes an unreacted amine group. In some embodiments, the polymethine cyanine dye is selected from Cy5, Cy7, meso-substituted Cy5, and meso-substituted Cy7.

In some embodiments, the polymethine cyanine dye has a percentage conjugation of 0.25%. In other embodiments, the polymethine cyanine dye has a percentage conjugation of 0.75%. In still other embodiments, the polymethine cyanine dye has a percentage conjugation of 0.75% to 3%. In other embodiments, the polymethine cyanine dye has a percentage conjugation of more than 3% to 5%.

The length of the polyamino acid may be varied. Thus, in some embodiments, the polyamino acid polymer comprises 100 to 1000 lysine residues. In other embodiments, the polyamino acid polymer comprises 200 to 800 lysine residues. In yet other embodiments, the polyamino acid polymer comprises 350 to 450 lysine residues.

In some embodiments, the percentage conjugation of the magnetic resonance imaging moiety is greater than 80% and less than 99%. In other embodiments, the percentage conjugation of the magnetic resonance imaging moiety is greater than 90% and less than 99%.

In another aspect, provided herein are imaging methods using the disclosed image-enhancing agents. In one embodiment, the imaging methods comprising the steps of, (a) administering the image-enhancing agent, to a subject; and (b) imaging the subject using a magnetic resonance technique, an optical imaging technique, or a combination thereof. In one variation, the imaging step comprises a magnetic resonance technique and an optical imaging technique, which both occur in a closed surgical field. In an alternative variation, the imaging step comprises a magnetic resonance technique and an optical imaging technique both occurring in an open surgical field. Where the disease tissue is tumor tissue, the imaging step comprises visualizing the tumor tissue using optical imaging techniques.

Timing the imaging step may vary, occurring from 30 minutes following administration of the image-enhancing agent. In some embodiments, the imaging step occurs more than 6 hours after administration of the agent. In other embodiments, the imaging step occurs 10 hours after administration of the agent. In yet other embodiments, the imaging step occurs at 12 hours after administration of the agent.

In some embodiments, the optically measured target to background ratio for the disease tissue relative to the non-diseased tissue is greater than 2:1. In other embodiments, optically measured target to background ratio for the disease tissue relative to the non-diseased tissue is greater than 4:1.

In another aspect, disclosed herein are method for managing disease tissue in a body comprising, (a) distinguishing disease tissue from non-diseases tissue in the body using the image-enhancing agent of claim 1; and (b) treating the disease tissue. In some embodiments of this aspect, the treating step comprises the step of excising at least the disease tissue. In other embodiments, the treating step comprises administrating a radiotherapuetic agent or a chemotherapeutic agent to at least the disease tissue identified by the image-enhancing agent.

The disclosed methods may be employed in an open surgical field, a closed surgical field, or a combination of open and closed surgical fields. Thus, in some embodiments, the distinguishing step and the treating step both occur in a closed surgical field. In other embodiments, either the identifying step or treating step occurs in an open surgical field. In some specific embodiments, the identifying step includes using a magnetic resonance device in a closed surgical field followed by using an optic device in an open or a closed surgical field.

### BRIEF DESCRIPTION OF THE DRAWINGS

The present invention will be more fully understood from the following description of preferred embodiments, when read together with the accompanying drawings, in which:

FIG. 1 is a chart showing the measured tumor to background values for the Gd-PL-Cy5 image-enhancing agents at different dosing conditions and time points. Error bars represent standard deviation.

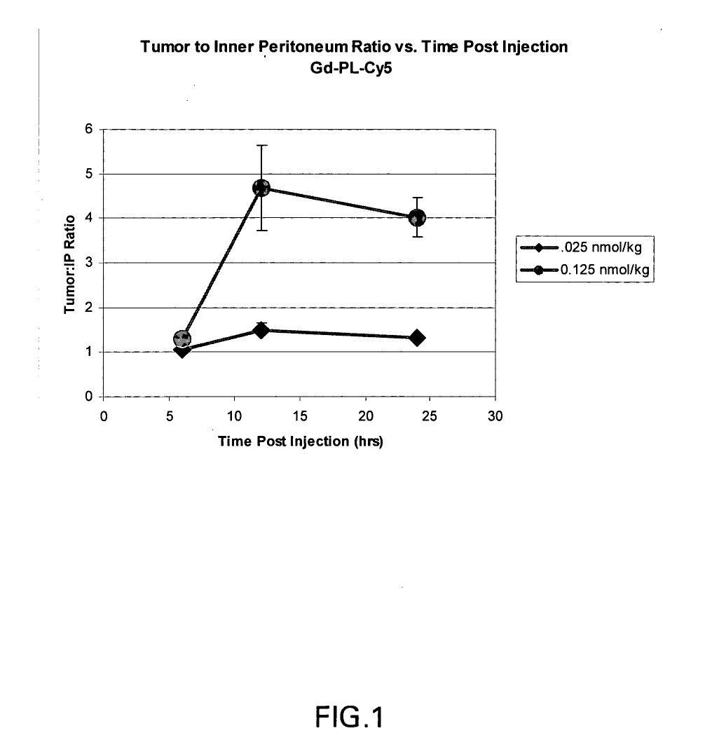

FIG. 2 Positive tumor labeling is detectable through the skin and fur of the animal treated with Gd-PL-Cy5. The highest dosing condition (125 nm dye/kg, corresponding to 125 nm polymer/kg) and longest time point post injection (24 h) yielded the brightest fluorescence. 2A is a color camera image; 2B is a near-infrared (NIR) camera image; and 2C is a merged image from both color and NIR camera.

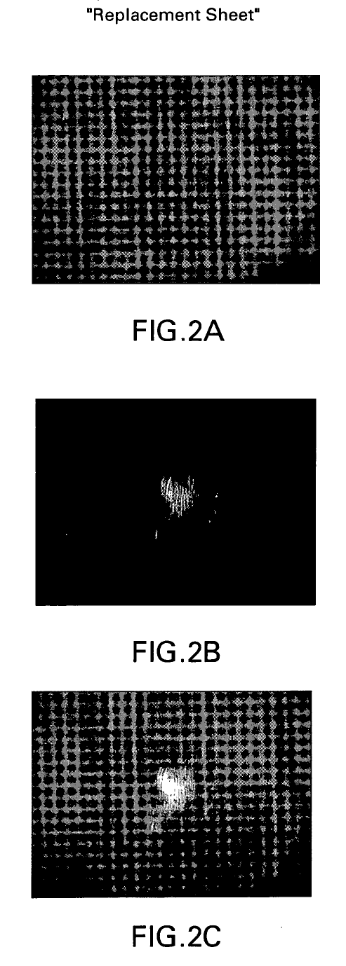

FIG. 3 Optical imaging results in the model animal in which the subject was imaged at the 12-hour time point and highest dosing conditions with Gd-PL-Cy5. The primary tumor had grown into the peritoneum. 3A is a color camera image; 3B is a Near-infrared (NIR) camera image; and 3C is a merged image from both color and NIR camera.

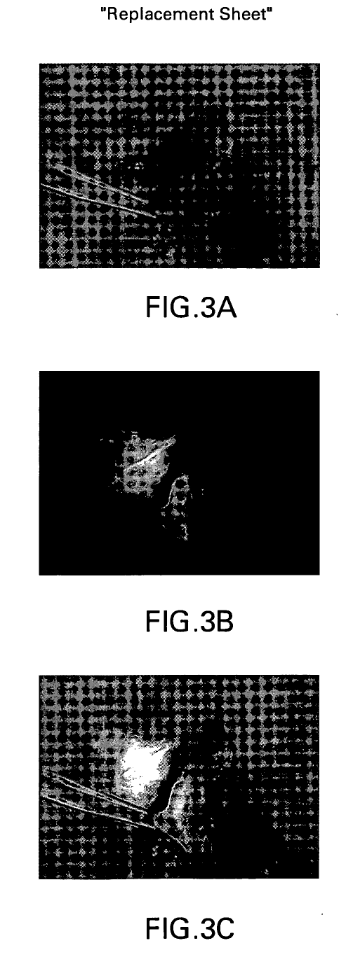

FIG. 4 shows the periphery of a tumor-bearing mammary gland after the application of hematoxylin and eosin staining, which verify image-enhancing agent localization in tumor tissue. Regions of dense granularity (the left half) show greater nuclear density, revealing tumor tissue. The less dense regions (the right half) demonstrate typical staining patterns for normal glandular epithelial tissue.

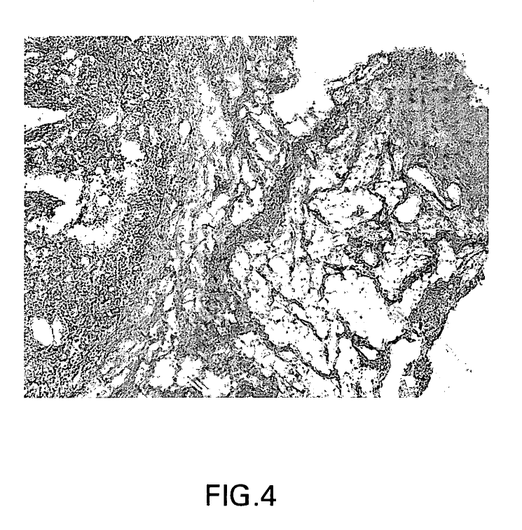

FIG. 5 shows fluorescent microscopy detection of the Gd-PL-Cy5 in the tumor margin. Tissue sections were stained for DAPI, which stains cell nuclei, and proliferating cell nuclear antigen (PCNA) for tumor cell identification. 5A is a black and white image of nuclear staining (DAPI). 5B is a black and white image of tumor cells staining (PCNA), in which only a portion of the stained nuclei (as seen in 5A) co-stain for PCNA. 5C: is a black and white image of Gd-PL-Cy5, in which the Gd-PL-Cy5 does not colocalize with the tumor cells (as seen in 5B). Gd-PL-Cy5 is detected in the area of normal mammary cells adjacent to the tumor cells (tumor margin).

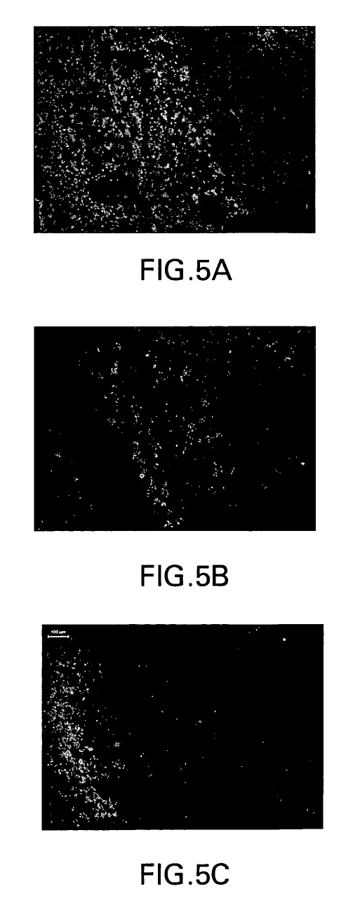

FIG. 6 is a chart showing the HPLC profile for two image-enhancing agents with different dye loading values.

FIG. 7A depicts MR imaging results in which tumor (positioned at the top of the animal to left of center) enhancement is shown 24 hours post injection of an image-enhancing agent loaded with 1 Cy5 dye per polymer. FIG. 7B depicts MR imaging results in which tumor (positioned at the top of animal to left of center) enhancement 24 hours post injection using an image-enhancing agent loaded with 16 Cy5 dyes per polymer.

FIG. 8 is a chart showing the dynamic uptake slope for image-enhancing agents with varying amounts of optical image moieties.

FIG. 9 is a chart showing the change in signal over time.

### DETAILED DESCRIPTION

Definitions

To more clearly and concisely describe and point out the subject matter of the claimed invention, the following definitions are provided for specific terms that are used in the following description and the claims appended hereto.

Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

As used herein, the term “chelator” refers to organic compounds in which the compounds form more than one coordination bond with metals in solution.

As used herein, the term “closed surgical field” refers to surgical fields accessed using non-invasive surgical techniques or minimally invasive techniques such as endoscopic techniques and devices that provide a physician access to the putative diseased tissue without requiring large incisions to expose the surgical field.

As used herein the term “disease management” refers to medical attention to disease conditions that may be facilitated using information derived from magnetic resonance imaging, optical imaging, or a combination of magnetic resonance imaging and optical imaging. Disease management includes decisions made by medical professionals regarding the course of treatment for a subject afflicted with a disease, including without limitation, the success or failure of a treatment, the status of the disease tissue, and/or whether chemical, radiation or surgical intervention is indicated. Where surgical or other non-systemic intervention is indicated, disease management also includes spatial localization of the disease tissue.

As used herein, the terms “dose” and “dosage” refers to the amount of agent delivered to the subject measured by the ratio of the nanomoles of the disclosed image-enhancing agent per kilogram body weight of the subject.

As used herein, the term “dye loading” refers to the number of dye molecules covalently bounds to the residues comprising the polymer backbone. Specifically, “low dye loading” indicates a percentage conjugation of the optical imaging moiety of between 0.25% to 0.99%. “Medium dye loading” indicates a percentage conjugation of the optical imaging moiety between 1% to 2.99%. Furthermore, “high dye loading” indicates a percentage conjugation for the optical imaging moiety that is more than 3%.

As used herein, the term “macromolecule” generally refers to chemical species comprising multiple repeat units that include functional groups that can react with dyes or metal chelators to form covalent bonds. The macromolecule described herein may include a polyamino acid polymer comprised of a single or multiple amino acids (e.g., lysine alone or lysine in combination with glutamic acid or aspartic acid).

As used herein, the term “magnetic resonance imaging moiety” refers to any chemical species that enhances a magnetic resonance image including, without limitation, paramagnetic and superparamagnetic ions.

As used herein, the term “open surgical field” refers to surgical techniques employing one or more incisions greater than 3 inches each that provide the physician with access to the putative diseased tissue.

As used herein, the term “optical imaging moiety” refers to fluorescent dyes with high extinction coefficients (>75,000), with quantum efficiencies greater than 0.1 and emissions above 600 nm.

As used herein, the term “paramagnetic ion” refers to the transition metals and lanthanide ions that that influence the relaxation time of water protons. Representative paramagnetic ions include, but are not limited to: gadolinium (III), dysprosium (III), holmium (III), europium (III), iron (III), or manganese (II).

As used herein, the terms “percentage conjugation” and “degree of conjugation” refer to the level of conjugation of a pendant group (i.e., a magnetic resonance imaging moiety or a optical imaging moiety) per reactive group on a macromolecule. One may control the level of percentage conjugation by varying the stoichiometric amount of the pendant group(s). Thus, for example, when the macromolecule is a polyamino polymer of 100 residues, the total available reactive groups (e.g., amine groups) for the polymer is 100, and 90% conjugation of the magnetic imaging moiety means that 90 of the available reactive groups are covalently bound to a pendant group.

As one of ordinary skill art would readily appreciate, the total percentage conjugation for the appended moieties may not exceed the total number of reactive groups available in a particular macromolecule. Although the combined percentage conjugation for the two appended moieties (i.e., magnetic resonance imaging moiety and the optical imaging moiety) may not exceed 100%, they need not necessarily add up to 100% (i.e., some reactive groups are not covalently bound to an appended moiety). Preferential tumor uptake of the image-enhancing agent is diminished at or near 100% conjugation of reactive groups. Accordingly, at least 1% of the monomeric units making up the polymer backbone include reactive groups that are not covalently bound to an appended moiety.

As used herein the term “pharmaceutically acceptable carrier” refers to those compounds which are, within the scope of sound medical judgment, suitable for use in contact with the tissues of a subject without undue toxicity, irritation, allergic response, and the like, commensurate with a reasonable benefit/risk ratio, and effective for their intended use, as well as salts and biocompatible derivatives of those compounds.

As used herein, the phrase “target to background ratio” generally refers to the mean signal intensity displayed by the target tissue over the mean signal intensity displayed by the background tissue.

As used herein, the term “treating” refers to the administration of curative or palliative measures to ameliorate the disease condition, including excision of the diseased tissue and, optionally, marginal tissue surrounding the perimeter of the diseased tissue using surgical techniques including, for example, ablation or resection.

As used herein, terms “tumor” and “tumor tissue,” refer to neoplastic cell growth and proliferation in a body.

As used herein, the term “tumor margin” refers the distance between the edge of the excised tissue and the presence of cancerous cells, as normally determined by histological evaluation. Chances of reoccurrence of the disease decrease with increasing tumor margin.

### SPECIFIC EMBODIMENTS

Disclosed herein are image-enhancing agents and methods of using the disclosed image-enhancing agents to detect and manage disease conditions. The image-enhancing agents comprise a magnetic resonance imaging moiety and an optical imaging moiety. These dual functional units provide flexibility to the user (e.g., a surgeon). Thus, for example, the disclosed image-enhancing agents can localize and characterize disease tissue preoperatively using MR techniques and later localize and characterize the same disease tissue intraoperatively using optical detection methods.

The backbone of the disclosed imaging agents may comprise a macromolecule, including an amino acid polymer. In some embodiments, the amino acid polymer is comprised of a single amino acid residue, for example, lysine. In alternative embodiments, the amino acid polymer is comprised of several different amino acid residues, for example, lysine, glutamine, arginine; combinations of lysine, glutamine, and arginine; and sub-combinations of lysine, glutamine, and arginine. In some embodiments, the macromolecules described herein have a molecular weight greater than about 10 kDa. In other embodiments, the macromolecules have a molecular weight greater than about 50 kDa. In still other embodiments the macromolecule has a molecular weight greater than about 200 kDa.

The polymeric backbone of the imaging agent may vary in length. Thus, in some embodiments the polymeric backbone may consist of about 100 to about 1000 residues. In other embodiments, the polymeric backbone consists of about 200 to about 800 residues. In yet other embodiments, the polymeric backbone consists of about 350 to about 450 residues.

In the disclosed image-enhancing agents, the magnetic resonance imaging moiety may be any chemical species that enhances a magnetic resonance image including, without limitation, paramagnetic and superparamagnetic ions. Representative paramagnetic ions may include, but are not limited to: gadoliniurri (III), dysprosium (III), holmium (III), europium (III), iron (III), or manganese (II).

The magnetic resonance imaging moiety may be covalently bound to the polymeric backbone using a chelator. Representative chelators include, without limitation, diethylene triamine pentaacetic acid (DTPA); ethylene diamine tetraacetic acid (EDTA); 1,4,7,10-tetraazacyclododecane-N,N′,N″,N′″-tetraacetic acid (DOTA); p-isothiocyanatobenzyl-1,4,7,10-tetraazacyclododecane-1,4,7,10-tetraacetic acid (p-SCN-Bz-DOTA); 1,4,7,10-tetraazacyclododecane-N,N′,N″-triacetic acid (DO3A); 1,4,7,10-tetraazacyclododecane-1,4,7,10-tetrakis(2-propionic acid) (DOTMA); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (NOTA); 1,4,8,11-tetraazacyclotetradecane-N,N′,N″,N′″-tetraacetic acid (TETA); triethylene tetraamine hexaacetic acid (TTHA); trans-1,2-diaminohexane tetraacetic acid (CYDTA); 1,4,7,10-tetraazacyclododecane-1-(2-hydroxypropyl)4,7,10-triacetic acid (HP-DO3A); trans-cyclohexane-diamine tetraacetic acid (CDTA); trans(1,2)-cyclohexane dietylene triamine pentaacetic acid (CDTPA); 1-oxa-4,7,10-triazacyclododecane-N,N′,N″-triacetic acid (OTTA); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

In some embodiments, the percentage conjugation of the magnetic imaging moiety is greater than 70%, leaving up to 30% of the reactive groups available for the optical imaging moiety. In other embodiments, the percentage conjugation of the magnetic imaging moiety is greater than 80%, leaving up to 20% of the reactive groups available for the optical imaging moiety. In still other embodiments, the percentage conjugation of the magnetic imaging moiety is greater than 90%, leaving up to 10% of the reactive groups available for the optical imaging moiety. Not all the reactive groups, however, are conjugated to the magnetic resonance imaging and optical moieties. Conjugation of all or substantially all reactive groups leads to the loss of preferential tumor uptake. About 1% of reactive groups should remain free to give preferential tumor uptake. In some preferred embodiments, the chelator is DTPA and the magnetic resonance imaging moiety comprises gadolinium.

In some embodiments the emissions for the optical imaging moiety are higher than 650 nm. In some embodiments, the dyes are either water soluble or rendered water-soluble by the addition of one or more solubility enhancing moieties (e.g., polyhydroxyalkyls, polyethers, sugar moieties, charged groups, e.g., sulfonates, phosphates, and carboxylates). In some embodiments, the optical imaging moiety comprises a cyanine dye. A family of cyanine dyes is described in PCT/US2003/014632 application, which is incorporated herein by reference. Preferred optical imaging moieties comprise those members of the cyanine dye family that are derived from an indole moiety (e.g., Cy5.0, Cy7.0 or IR800).

In some embodiments the dye is selected from a cyanine dye of the following structure:

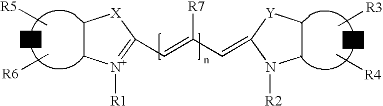

R¹ and R² are selected from C₁-C₁₀ alkyl, which may be substituted or unsubstituted;

wherein, groups R³, R⁴, R⁵ and R⁶ are attached to the rings Z₁ and Z₂;

n is an integer from 1-3;

Z₁ and Z₂ each represent the atoms necessary to complete a five or six member fused aromatic ring containing carbon atoms and optionally a heteroatom selected from O, N, or S;

X and Y are independently selected from a R⁸R⁹C, an oxygen, a sulfur, a selenium or —CH═CH—;

R³, R⁴, R⁵, R⁶, R⁸ and R⁹ are independently selected from the group consisting of hydrogen, substituted or unsubstituted C₁-C₆ alkyl, amino, substituted amino, acylamino, substituted acylamino, quarternary ammonium, phosphate, sulfate, sulfonate, fluoro, chloro, COOR¹⁰, OR¹⁰, and —(CH2)n-P, where R¹⁰ is substituted or unsubstituted and is selected from H, C₁-C₆ alkyl, P is amino, substituted amino, amido, substituted amido, carboxylate, sulfate, sulfonate, phosphate, hydroxyl, quarternary ammonium and groups reactive with amino, hydroxyl, carbonyl, carboxyl, phosphoryl and sulfhydryl groups on the polymer;

R⁷ is selected from H, C₁-C₂ alkyl, Cl, OR₁₁, where R₁₁, is an a C₁-C₆ alkyl, aryl or substituted aryl; and

at least one of the groups among R₁-R₉ is or substituted with a group reactive with amino, hydroxyl, carbonyl, carboxyl, phosphoryl, or sulfhydryl groups.

In some particular embodiments, the optical imaging moiety may be represented by Formulas I and II

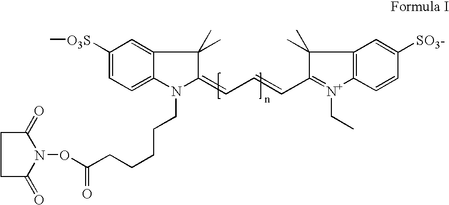

wherein n=1, 2, or 3 for Cy3, Cy5, or Cy7.

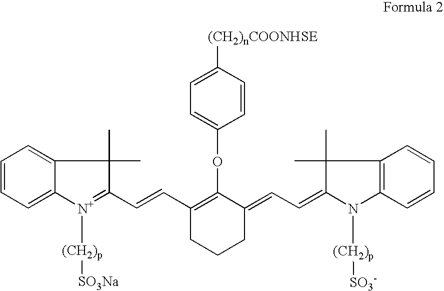

wherein p=3-5.

In another embodiment, the dye may be represented by the structure

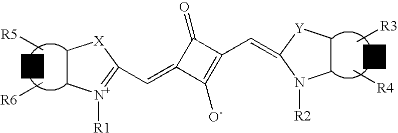

wherein, groups R³, R⁴, R⁵ and R⁶ are attached to the rings Z₁ and Z₂;

Z₁ and Z₂ each represent the atoms necessary to complete a five or six member fused aromatic ring containing carbon atoms and optionally a heteroatom selected from O, N or sulfur atom;

X and Y are independently selected from a R⁸R⁹C, an oxygen, a sulfur, a selenium or —CH═CH—;

R¹ and R² are selected from C₁-C₁₀ alkyl, which may be substituted or unsubstituted;

R³, R⁴, R⁵, R⁶, R⁸ and R⁹ are independently selected from the group consisting of hydrogen, substituted or unsubstituted C₁-C₆ alkyl, amino, substituted amino, acylamino, substituted acylamino, quarternary ammonium, phosphate, sulfate, sulfonate, fluoro, chloro, COOR¹⁰, OR¹⁰, and —(CH2)n-P, where R¹⁰ is substituted or unsubstituted and is selected from H, C₁-C₆ alkyl, P is amino, substituted amino, amido, substituted amido, carboxylate, sulfate, sulfonate, phosphate, hydroxyl, quarternary ammonium and groups reactive with amino, hydroxyl, carbonyl, carboxyl, phosphoryl, and sulfhydryl groups on the polymer; and

at least one of the groups among R₁-R₉ is or substituted with a group reactive with amino, hydroxyl, carbonyl, carboxyl, phosphoryl, and sulfhydryl groups.

The dyes of structures described above may include one or more counter ions, which may be positive or negative to balance the formal charge (or charges) on the dye chromophore or on substituent groups. The nature of the counter ion is immaterial as long as it is pharrnacologically acceptable.

It is within the scope of the current invention to contemplate that one or more of the R groups described above may be modified to link other modifier such as those to modulate solubility, circulation, or other pharmacologically desirable properties.

In some embodiments, the percent conjugation of the optical imaging moiety is at least 0.25%. In other embodiments, the percent conjugation of the optical imaging moiety is at least 0.75%. In yet other embodiments, the percent conjugation of the optical imaging moiety is between about 1% to about 3%. In yet other embodiments, the percent conjugation of the optical imaging moiety is between about 4% and about 5%.

In some embodiments, the disclosed imaging agents are disposed in a pharmaceutically acceptable carrier which is, within the scope of sound medical judgment, suitable for use in contact with the tissues of a subject without undue toxicity, irritation, allergic response, and the like, commensurate with a reasonable benefit/risk ratio, and effective for their intended use, as well as salts and biocompatible derivatives of those compounds.

For both the magnetic resonance and optical imaging indications, the administration step may comprise introducing from about 5 nm/kg bw to about 150 nm/kg bw of the imaging agent to the subject. In some embodiments, about 125 nm dye/kg (corresponding to 125 nm polymer/kg) or 0.025 mmol Gd/kg (corresponding to 65 nm of polymer/kg) is administered to the subject. Modes of administration include injecting the imaging agent into the subject intravascularly, intraperitoneally, or intramuscularly. In some specific embodiments, the image-enhancing agent is directly administered into the particular tissue to be imaged (e.g., intra-tumorally).

MR detection may be performed using a 1.5T scanner (e.g., GE Signa). A T1 weighted spin echo sequence may be used (e.g., TE 9 msec and TR 250 msec). Images may optionally be acquired before administration of agent for reference and again later after administration (e.g., by intravenous injection) of the agent. MR images may be acquired minutes following injection and for many hours after (e.g., between 30 minutes following injection to up to 24 hours post-injection).

For the optical imaging methods, continuous imaging may be completed using any standard fluorescent imaging system. When the subject is a small animal a standard small animal fluorescent imaging system may be employed. Its components include two wavelength-isolated excitation sources, one generating visible light, and another generating NIR light. Simultaneous photon collection of color video and NIR fluorescence channels is achieved with optics that maintain separation of white light and NIR fluorescence channels. After computer-controlled (LabVIEW) camera acquisition using LabVIEW software, anatomic and functional images can be displayed separately and merged. All images are refreshed up to 15 times per second.

In some embodiments, the target to background ratio for the disease tissue relative to the non-diseased tissue using the disclosed image-enhancing agents in optical imaging applications is greater than 2:1. In other embodiments, the target to background ratio for the disease tissue relative to the non-diseased tissue optical imaging applications is greater than 4:1.

For optical imaging the signal to ratio background ratio is defined as the mean fluorescence intensity (target tissue)/mean fluorescence intensity (background tissue). For optical imaging methods, target to background ratio may be determined using computer-controlled (LabVIEW) camera acquisition via custom LabVIEW software. Anatomic (white light) and functional (NIR fluorescent light) images can be displayed separately and merged. A region of interest (ROI) maybe defined in both the tumor area and the background tissue area. Fluorescence emission from the ROI may be received at a near-infrared sensitive 12-bit CCD camera, in which the intensities range from 0-4095.

The optical detecting step or the magnetic imaging step may occur from between 30 minutes to 24 hours following administration of the image-enhancing agent. In some embodiments, the detection step occurs more than 6 hours after administration of the image-enhancing agent. In some other embodiments, the imaging step occurs more than 10 hours after administration of the image-enhancing agent. In yet other embodiments, the imaging step occurs at about 12 hours after administration of the image-enhancing agent.

For MR imaging methods target to background ratio is generally measured by taking a pair of images, one before administration of the image-enhancing agent, and one after administration of the image-enhancing agent. Thus, the target to background ratio may be determined by taking an MR image is taken before contrast agent injection and then after agent injection at a selected time. The change of image intensity between the two images for a given position in the image is the signal enhancement at the selected time. The tumor signal is the enhancement observed in a defined region of interest, ROI, in the tumor or a region of interest encompassing the entire tumor. The signal intensity value of an ROI in an MR image is given in the MR scanner display as an average of the intensities of the voxels in the ROI. Alternatively, the image voxel intensity values can be transferred to an image analysis program and the same ROI voxel averaging can be performed in that program application. Taking the difference of the signal intensities of an ROI between the two images gives the signal enhancement observed in that ROI. The tumor signal to background signal is the enhancement observed in the tumor compared to the enhancement observed in tissue that is near the tumor, usually muscle tissue. If there is to be significant time lapse between the two image acquisitions, a signal reference such as a tube of corn oil can be inserted adjacent to the tissues being imaged to normalize for possible drift in MR scanner parameters.

Using the imaging of disease tissue agents and techniques disclosed herein, a physician can delineate healthy tissue from disease tissue to selectively treat the disease tissue. Thus, a physician may administer to a subject the disclosed agents, image the subject using the methods of the invention, and manage the disease according to the results of the image.

In some embodiments, the disease management comprises use of information derived from magnetic resonance imaging, optical imaging, or a combination of magnetic resonance imaging and optical imaging obtained using the disclosed agents to spatially localize the diseased tissue. Where treatment is indicated, disease management may include decisions made by medical professionals regarding the course of treatment for a subject afflicted with a disease, including without limitation, the success or failure of a treatment, the status of the disease tissue, or whether chemical, radiation or surgical intervention is indicated.

Because the disclosed agents comprise both MR imaging moieties and optical imaging moieties, the imaging methods may include imaging the subject with devices that detect a paramagnetic contrast agents, devices that detect fluorescence, or both in combination. Thus some embodiments, the disease management methods may comprise administering the agents of the invention, MR detection, followed by optical detection. In some embodiments, the methods optionally include treating the disease tissue.

In some embodiments, one of the two detection methods occurs in a closed surgical field and the second detection method occurs in an open surgical field. For example, in one series of embodiments, the imaging agent is administered to the subject; the target tissue or organ is imaged using MR to initially determine the presence, location, or extent of the disease tissue; the surgical field is opened by an incision; and the target tissue or organ is then imaged using fluorescence.

In further embodiments, both detection methods may occur in a closed surgical field, imaging the target tissue or organ using MR, followed by optical imaging of the same target tissue or organ employing endoscopic devices and techniques. For example, in one series of embodiments, the imaging agent is administered to the subject; the target tissue or organ is imaged in a closed field using MR techniques; and the target tissue or organ is imaged in a closed surgical field using optical techniques. Optical imaging in a closed surgical field may be accomplished by measuring the level of fluorescence at the skin surface or through a bodily cavity (e.g., the colon, the esophagus, or the vaginal canal). Alternatively, optical imaging may be accomplished by creating an incision large enough to accommodate a cannula, introducing a fluorescent detector attached to an endoscopic device by, deploying the fluorescent detector to the location of the target tissue or organ, and measuring the fluorescence emitted by the target tissue or organ.

In some embodiments, the treating step comprises the step of excising (e.g., by ablation or resection) at least the disease tissue identified by the image-enhancing agent. In alternative embodiments, the treatment step comprises administrating a radiotherapuetic agent or a chemotherapeutic agent to at least the disease tissue identified by the image-enhancing agent. In yet other embodiments, the treating step comprises excising at least the disease tissue identified by the image-enhancing agent and administrating a radiotherapuetic agent or a chemotherapeutic agent. In each of the forgoing embodiments, the method of managing disease may comprise magnetic resonance imaging or optical imaging the disease tissue following treatment. Thus, for example, a managing disease method for cancer indications may comprise: imaging the disease tissue, treating the disease tissue, and subsequently imaging the same or remaining tissue to determine the efficacy of the treatment and to inform the subsequent course of treatment.

The agents disclosed herein detectably localize at disease tissue. Specifically, when the disease condition is cancer and the disease tissue is tumor tissue, an administered image-enhancing agent acts as a surrogate for margin of the tumor. For this particular embodiment, disease management may include imaging the disease tissue by administering a disclosed image-enhancing agent; imaging the disease tissue (either by a MR technique, an optical imagining technique, or both a MR technique and an optical imagining technique); followed by treatment of the disease tissue (e.g., through chemical, radiation or surgical means); and, optionally, imaging (either by a MR technique, an optical imagining technique, or both a MR technique and an optical imagining technique) the previously administered or subsequently administered image-enhancing agent to determine the efficacy of the treatment.

### EXAMPLES

Practice of the invention will be still more fully understood from the following examples, which are presented herein for illustration only and should not be construed as limiting the invention in any way.

### Example 1

Polylysine-DTPA-Gd Synthesis. Polylysine-DTPA-Gd was synthesized according to the method set forth in co-pending patent application U.S. Ser. No. 11/290,684, entitled “Conjugated Macromolecules,” which is incorporated by reference herein in its entirety.

Unless otherwise indicated, the following materials and methods were used in this Example 1. Poly(lysine), diethylenetriaminepentaacetic acid (>99%), isobutylchloroformate (>99%), triethylamine (>99%), gadolinium chloride, trisodium citrate and trinitrobenzene sulfonic acid (TNBSA, Pierce) were used as received; acetonitrile was distilled over CaH₂; 18Ω water was obtained from a Millipore four-stage filtration device. All reactions were performed on a Schlenk line under an N₂ inert atmosphere unless otherwise stated using glassware that had been pre-rinsed with 18 MΩ water. Prior to use for material purification, 5 kDa and 10 kDa MWCO ultrafilters (Amicon) were pre-rinsed with 18 MΩ water at 4000 rpm on a centrifuge (Sorvall RC-5B Superspeed Centrifuge, equipped with swinging bucket rotor). HPLC analysis was performed using a Dionex LC25 chromatography oven fitted with an Agilent Zorbax GF250 column, an AD25 absorbance detector and a GP40 gradient pump eluting with a potassium phosphate buffer (pH 7, 20 mM, 165 mM NaCl).

A sample of DTPA (3.38 g, 8.7 mmol) and acetonitrile (46 mL) was degassed for 20 minutes. Upon addition of NEt3 (6.20 ml, 43.9 mmol), the reaction mixture was heated to 60° C. for 1 hour with stirring. The resultant clear, colorless solution was then transferred by syringe to a three-necked flask, equipped with a mechanical stirrer, and cooled to −40° C. An acetonitrile solution (23 mL) of i-BuCOCl (1.25 mL, 9.7 mmol) was then added to the reaction mixture at a rate of 1.2 ml.min⁻¹ over the course of 20 minutes. The reaction mixture was allowed to stir for 1 hour at 45° C., resulting in the gradual formation of a white precipitate, at which point stirring was halted.

Under an inert atmosphere, one neck of the reactor was equipped with a septum fitted with a Teflon tube that was inserted into the reaction mixture. The DTPA reagent was maintained at −40° C., and then pumped (4.4 ml.min⁻¹ for 17 minutes) to a second reactor containing a clear, colorless aqueous NaHCO₃ (0.1 M, pH 10)/NaCl (2 M, 2.69 g, 46 mmol) solution (23 mL) of poly(lysine) (0.25 g, 1.2 mmol M_{w}=84,000 g.mol-1, DP=402) at ambient temperature. The resulting cloudy biphasic reaction mixture stirred vigorously in a baffled reactor for 16 hours at which point stirring was stopped, the reaction mixture transferred to a separatory funnel and then allowed to settle into two clear colorless phases. The lower aqueous layer was separated and added to an aqueous NaHCO₃ (0.1 M, pH 10)/NaCl (2 M, 2.69 g, 46 mmol) solution (23 mL) to afford a clear colorless solution of the crude product.

The resultant crude reaction mixture was purified by ultrafiltration (4× Amicon Ultrafilters, 10K MWCO, 2000 rpm), and washed with five cycles of distilled water (4×12 mL, 18 MΩ) to remove low MW byproducts, as verified by GPC, providing the product Poly(lysine-DTPA) as a clear colorless DI solution, in an 84% yield, with a 98% purity (HPLC) and 97±1.4% conjugation, MW using Multiangle Laser Light Scattering (“MALLS”)=208 kDa.

### Example 2

Direct Gadolinium Labeling of Poly(lysine-DTPA) in the Synthesis of Poly(lysine-DTPA-Gd). The crude PL-DTPA product described above was treated with GdCitrate (44 mL, 0.3 M) and allowed to stir at ambient temperature for 16 hours. The crude reaction mixture was then concentrated to 40 mL volume by ultrafiltration (4× Amicon Ultrafilters, 10K MWCO, 2000 rpm), and washed with five cycles of distilled water (4×12 mL, 18 MΩ) to remove low molecular weight by-products, as verified by GPC, providing the product Poly(lysine-DTPA-Gd) as a clear colorless DI solution, in a 71% overall yield, with 99% purity (GPC) and 97±1.4% conjugation, MW (MALLS)=270 kDa.

Table 1 shows the chemical names of several dyes that were used in the following Examples.

TABLE 1

| | |
|---|---|
| Cy3 | 2-[5-[1-(5-carboxypentyl)-1,3-dihydro-3,3-dimethyl-5-sulfo- |
| | 2H-indol-2-ylidene]-tri-1-enyl]-3,3-dimethyl-5-sulfo-1- |
| | ethyl-3H-indolium, inner salt, sodium salt |
| Cy5 | 2-[5-[1-(5-carboxypentyl)-1,3-dihydro-3,3-dimethyl-5-sulfo- |
| | 2H-indol-2-ylidene]-penta-1,3-dienyl]-3,3-dimethyl-5-sulfo- |
| | 1-ethyl-3H-indolium, inner salt, sodium salt |
| Cy7 | 2-[7-[1-(5-carboxypentyl)-1,3-dihydro-3,3-dimethyl-5-sulfo- |
| | 2H-indol-2-ylidene]-hepta-1,3,5-trienyl]-3,3-dimethyl-5- |
| | sulfo-1-ethyl-3H-indolium, inner salt, sodium salt |
| Cy5.5 | 2-[5-[1-(5-carboxypentyl)-1,3-dihydro-3,3-dimethyl-6,8- |
| | disulfo-2H-benzo[e]indol-2-ylidene]-penta-1,3-dienyl]- |
| | 3,3-dimethyl-6,8-disulfo-1-ethyl-1H-benzo[e]indolium, inner |
| | salt, sodium salt |
| IR800 | [2-[2-[3-[(1,3-dihydro-3,3-dimethyl-1-[4-sulfobutyl]- |
| | 2H-indol-2-ylidene)ethylidene]-2-[4-(carboxyalkyl)]-1- |
| | cyclohexen-1-yl]ethenyl]-3,3-dimethyl-1-[4- |
| | sulfobutyl]indolium salt |

### Example 3

Synthesis of Gd-DTPA-PL-Cy. In these examples the following reagents were used: the Cy5, Cy5.5, Cy7, NHS esters (GE Healthcare); IR800 NHS ester (LiCOR); all other materials were obtained from Aldrich.

Step 1. Conjugating the dye to the polymer. Gd-DTPA polylysine (˜4-6 μmol free lysines) was dissolved in 0.4 ml of 0.1 M sodium carbonate at pH 8.6-8.8 (adjusted by addition of sodium carbonate). NHS ester of dye was dissolved in DMSO (1 mg/30 μl) and a portion of this solution containing 0.05 μmol of active dye-NHS ester/μmol of free lysine (calculated based on % chromophore and % NHS ester information provided by the supplier) was added to the Gd-DTPA polylysine solution.

Step 2: Purification. The mixture was vortexed and stored at room temperature overnight in the dark with occasional vortexing. The mixture was then diluted with water to reduce the percentage of DMSO to less than 5% and then filtered on an Amicon filter (MW cutoff 10,000) at 4° C. The residue was washed repeatedly with water (10-15 ml) until the filtrate was almost colorless (at least 3 washes). The residual material was purified on Superdex 200, 10×300 mm column using water as eluant at a flow rate of 0.5 ml/minute.

Step 3: Fractionation. The product was collected in fractions, which were pooled into a smaller number of fractions based on elution profile. These fractions were analyzed by: UV for dye concentration; HPLC for presence or absence of low molecular weight impurities; and ICP-MS for Gd analysis. HPLC analyses were performed on Agilent Zorbax GF-250, 4.6×250 mm column, particle size 4 μm optical imaging applications using 1× PBS as elution buffer at 0.5 ml/min, as well as on Tosoh Biosciences G-DNA-PW 7.8×300 mm column, particle size 10 μm using 200 mM NaCl at 0.5 ml/min. Dye loading was calculated using UV absorbance and Gd analysis.

### Example 4

Synthesis of Gd-DTPA-PL-Cy dye with medium or high dye loading. Gd-DTPA polylysine (˜3 μmol free lysines) was dissolved in 0.4 ml of 0.1 M sodium carbonate at pH 8.6-8.8 (adjusted by addition of sodium carbonate). NHS ester of dye was dissolved in DMSO (1 mg/30 μl) and a portion of this solution containing 0.6-1.2 μmol (0.6 for medium and 1.2 for high) of active dye-NHS ester/μmol of free lysine (calculated based on % chromophore and % NHS ester information provided by the supplier) was added to the Gd-DTPA polylysine solution. The product was purified and fractionated as described in Steps 2 and 3 in Example 2.

### Example 5

Optical Imaging Using Tumor Rat Model. Step 1. Introduction of Tumor Cells into Animal. Intra-mammary gland tumor injection is a survival surgery. All procedures are performed with the animal lying on a circulating water pad. With the anesthetized rat lying on its back, the sterile prepped ventral abdominal skin is picked up, just above the left groin (inguinal) area and a small incision is made with scissors being careful to avoid the underlying peritonium. The fourth mammary gland is easily visible, lying on the dorsal surface of the skin directly on top of a junction point for the mammary artery and vein. Using a 1 cc syringe, 10⁶ cells (0.1 ml) were injected directly into the gland. Only one mammary gland per rat was inoculated with tumor cells. The skin wound was closed with surgical staples. Surgical staples were removed 3 days after insertion.

Animals are treated with test agents and imaged 7 days after tumor injections. Test agents are administered via a tail vein injection (100-500 μl). For MR studies, animals received a pre-injection scan, followed by post-injection scans. Images were obtained post injection at approximately 4-minute intervals following injection for up to 30-40 minutes. In some experiments images were again taken at 24 hours after injection. For optical studies, animals were imaged at 0.5, 6, 12, and 24 hours.

Step 2. Agent Administration. After approximately 8 days following tumor cell implantation (the tumor size reached about 5-10 mm diameter) one of several imaging agents listed in Table 2 was administered to the animal at the corresponding dosage indicated in Table 2.

Step 3. Surgical Resection. Surgical tumor resection is a non-survival surgery. Tumors were resecteded when they reached a diameter in the range of 5-10 mm in diameter. With the anesthetized rat lying on its back, the ventral abdominal skin is picked up, just above the left groin (inguinal) area and a small incision is made (at or near the site of tumor inoculation) with scissors being careful to avoid the underlying peritonium. The tumor, growing in the mammary gland, stays contained in this tissue and is easily removed. The tumor was held with a pair of forceps and using a scalpel is removed from the surrounding connective tissue. After the tumor was removed, a midline incision, through the peritoneum was made, other organs were observed and imaged, and sampled for histological and biodistribution studies.

Step 4. Data Capture. Images were captured for later evaluation of the relative intensity of fluorescence in the kidney, liver, bladder, muscle etc. Samples of blood and urine were acquired and stored, the animal was sacrificed, and the clearance organs were removed from animal and frozen in a slurry of dry ice and 2-methylbutane. The tissues were then placed in an embedding medium (OCT, optical cutting temperature) and frozen at −80° C. Blood and urine samples were analyzed for concentration of agent at this time point, and for evidence of metabolism of the administered agent.

### Example 6

Histological evaluation. 10-micron tissue sections were cut using a cryotome. Immunohistochemistry was performed using a mouse monoclonal PCNA antibody (Sigma) as follows: slides were washed in PBS, blocked with normal donkey serum, labeled with primary antibody (1:1000) and then in mouse anti donkey secondary (1:200). Cover slips were mounted using AntiFade Gold+DAPI (Molecular Probes). DAPI stains for nuclei in the tissue samples and PCNA stains against the proliferating cell nuclear antigen, which is a marker for tumorgenic cells. Fluorescent images with Cy5-PL, DAPI, and PCNA were acquired and stored for further analysis.

After the entire tissue sample was imaged, and regions of interest were documented, the samples were stained using H&E staining for correlation of the distribution of these target specific stains in comparison with art-recognized staining patterns for tumor tissue.

### Example 7

Statistical Analysis of Optical Imagine Results. To reduce the large number of variables in this problem and one polymer length (˜400 residues) was chosen at a high percentage conjugation (˜96%) for the MR imaging moiety (Gd-DTPA) with one dye per molecule. In one set of experiments, a tetra-sulfonated dye (Cy5.5-NHS-Ester) was then compared with a bis-sulfonated dye (Cy5-NHS-Ester).

The impact of dose, the class of cyanine dye, and time post administration on the measurement of tumor uptake in comparison to a background tissue were evaluated. The results were compared to those obtained with mock injections and negative control animals. Three time points were chosen for evaluation. The results of imaging studies in 44 experimental subjects comparing the performance of the PL-Cy5, PL-Cy5.5, and Cy5 agents alone as shown in Table 2. This Table reports the average, standard deviations, and number of subjects in each test condition.

TABLE 2

| | | | Time (hours) | | | | |
|---|---|---|---|---|---|---|---|
| Vector | Dose (nmol/kg) | Data | 0.5 | 6 | 12 | 24 | Total |
| PL-Cy5 | 25 | Average of | | 1.05 | 1.50 | | 1.36 |
| | | Tumor/Bkgnd | | 1.31 | | | |
| | | StdDev Tumor/Bkgnd | | | 0.53 | | 0.36 |
| | | | | | 0.20 | | |
| | | N | | 1 | 3 | 3 | 7 |
| | 125 | Average of | | 1.35 | 4.69 | | 3.08 |
| | | Tumor/Bkgnd | | 4.02 | | | |
| | | StdDev Tumor/Bkgnd | | | 2.74 | | 2.04 |
| | | | | | 1.67 | | |
| | | N | | 1 | 2 | 3 | 6 |
| PL-Cy5 Average of Tumor/Bkgnd | | | | 1.20 | 2.78 | | 2.48 |
| | | | | 2.67 | | | |
| PL-Cy5 StdDev Tumor/Bkgnd | | | | 0.21 | 2.25 | | 1.85 |
| | | | | 1.83 | | | |
| PL-Cy5 N | | | | | | | 13 |
| Cy5 free dye | blank | Average of | | 1.17 | 1.18 | | 1.17 |
| | | Tumor/Bkgnd | | 1.17 | | | |
| | | StdDev Tumor/Bkgnd | | 0.14 | 0.11 | | 0.11 |
| | | | | 0.13 | | | |
| | | N | | 3 | 4 | 4 | 11 |

| | | | | |
|---|---|---|---|---|
| Cy5 free dye Average of Tumor/Bkgnd | 1.17 | 1.18 | | 1.17 |
| | 1.17 | | | |
| Cy5 free dye StdDev Tumor/Bkgnd | 0.14 | 0.11 | | 0.11 |
| | 0.13 | | | |
| Cy5 free dye N | 3 | 4 | 4 | 11 |

### Example 8

Tumor Animal Model. The experiment requires a clinically relevant tumor model for angiogenesis as well as a tumor that invades into the surrounding epithelia to generate a sample that is suitable for histological evaluation. A rat mammary tumor line was chosen (MAT-B-III) and 2×10⁶ cells were injected into the mammary gland and tumors were allowed to grow for 7-9 days. The imaging agent was administered to animals with palpable tumors, and returned to their cages until the appropriate time point. At specified times post injection (0.5, 6, 12, and 24 hours) the animal was put under general anesthesia, the tumor was exposed and imaged, and the entire gland was saved for histological evaluation. The peritoneum was then cut and a visual inspection of the clearance organs was performed.

Images were captured for later evaluation of the relative intensity of fluorescence in the kidney, liver, bladder, muscle etc. Samples of blood and urine were acquired and stored, the animal was sacrificed, and the clearance organs were removed from animal and frozen in a slurry of dry ice and methylbutane. The tissues were then placed in an OCT embedding medium frozen at −80° C. Blood and urine samples were then analyzed for concentration of agent, and for evidence of metabolism of the administered agent.

### Example 9

Optix Fluorescence. Dynamic fluorescence measurements were done with Mat B rat adenocarcinoma tumor model using subcutaneous tumor implantation. An ART Optix fluorescence imaging system was used in which a chosen region of animal is scanned with a 635 nm laser and the emitted fluorescence at 665 nm is collected with a lens, passed through a 670 nm bandpass filter, and directed into a photomultiplier detector.

Tumors were about 1 cm in diameter. The fluorescence signals were obtained by scanning over the entire tumor in an ROI of about 30×30 mm. For analysis emission photon counts were taken over a region of 6×4.5 mm centered on the brightest group of pixels of the tumor.

Experimental signal scaling factors associated with input light and fluorescent light collection efficiency were normalized as follows: Where S(t) is the fluorescence signal at time t post injection and S₁ is the signal immediately after agent injection, and C₁ is the circulating concentration of dye agent in the blood, and □₁ is a scaling factor encompassing factors such as incident light, geometric factors, tissues scattering and absorption, and fractional blood volume. Then
S₁=α₁C₁

To determine the permeability we must eliminate the scaling factor □₁, by taking a sequence of intensity measurements after the first measurement as the agent accumulates in the tumor through transendothelial transport:

$$\begin{matrix}\frac{S ( t ) - {S}_{1}}{{S}_{1}} = \frac{{α}_{1} [ C ( t ) - {C}_{1} ]}{{α}_{1} {C}_{1}} \\ = \frac{{k}_{1} \times {C}_{1} \times t}{{C}_{1}} \\ = {k}_{1} \times t\end{matrix}$$

Where k₁ is the agent uptake rate constant averaged over the volume of interest. Thus, k₁ is directly related to tumor permeability, the permeability surface area product. Hence, determining the signal slope, normalized to the initial signal after injection, gives a measure of the desired parameter, tumor endothelial permeability of the agent tested.

MR Examples. The agent used in the following examples was a 400-residue polylysine polymer in which about 96% of the residues were conjugated with gadolinium through a DTPA chelator. The available free lysines on this polymer were conjugated with a cyanine optical dye, specifically Cy5, Cy7, or IR-800 at various levels of dye loading. The agents where characterized by HPLC relative to standard Gd-PL constructs for changes in polymer conformation. The retention times in a BioSep column for these constructs did not vary from previously observed times for the same agents without the dye component, indicating that the polymer did not undergo coiling during the dye conjugation step.

MR imaging was performed using 1.5 Tesla on a GE Signa scanner adapted with a small-animal receiver coil. The animal tumor model was a rat mammary adenocarcinoma, Mat B, in female Fisher rats. Imaging experiments were performed when tumor size attained approximately a 1 cm diameter.

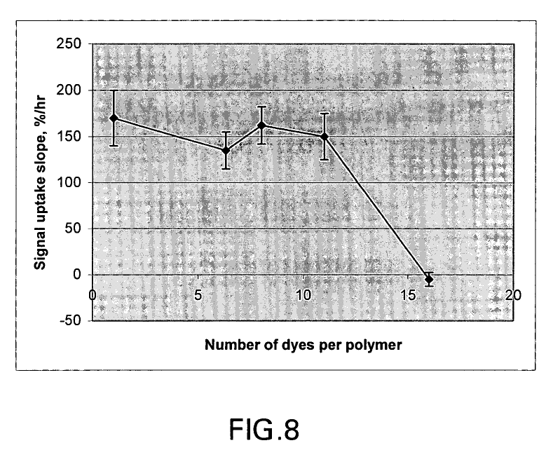

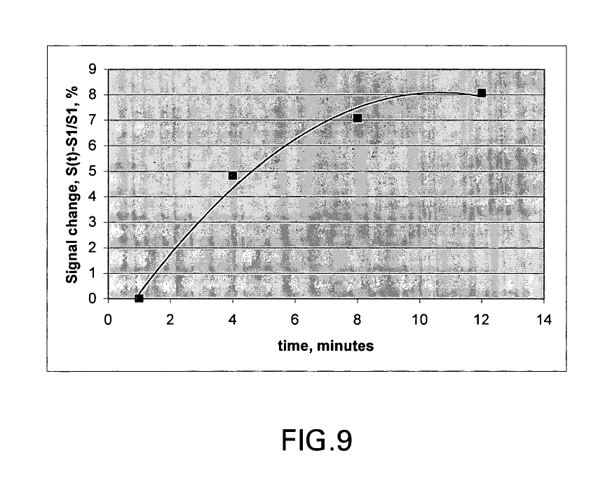

All animals were anesthetized; T1 weighted spin echo images were obtained and the animals were then injected with the dual agent at a dose of 0.025 mmoles Gd/kg (corresponding to 65 nm polymer/kg). Images were obtained post injection at approximately 4-minute intervals following injection for up to 30-40 minutes. In some experiments images were again taken at 24 hours after injection. Between 1 and 16 fluorescent dye molecules were appended to the polymer for the following experiments. As shown in FIG. 9, surface tumor fluorescence signal change of Gd-DTPA₃₈₆-PLL₄₀₂-Cy5₁ as a function of time, in first 12 minutes post tail vein injection (0.025 mmole Gd/kg, corresponding to 66 nm cy5/kg). The initial temporal rise in fluorescence signal with an apparent initial signal slope change of 110%/hr corresponds to MRI signal slope determinations in this tumor model of ˜150% per hour (FIG. 8).

### Example 10

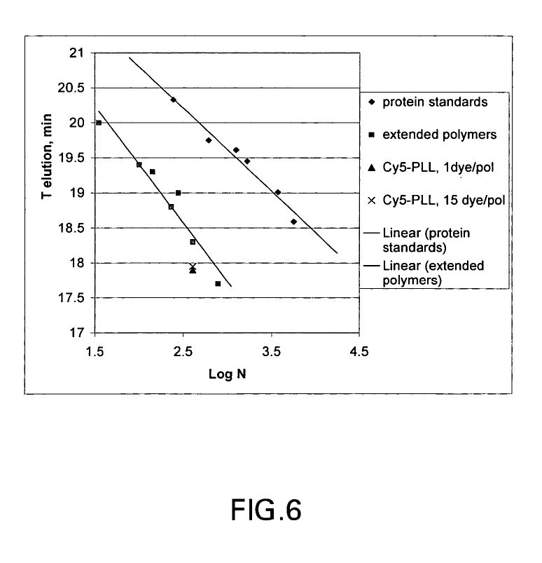

HPLC Retention Times. Shown in FIG. 6 is the HPLC retention times of both high and low dye load constructs. For both types, the unfolded, extended conformation seems to be maintained: The elution time is slightly below the expected time for 400 mer constructs of such a conformation, the blue lower line. Folding reactions result in an increased retention time and movement of the elution time up toward the globular protein line, upper red line. As shown in FIG. 6, the elution time of the image-enhancing agents fall near expected time for extended, uncoiled polymers.

### Example 11

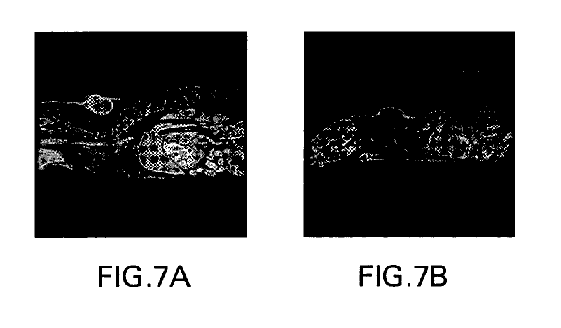

MR Imaging with Variable Dye Loading. FIG. 7B shows an MR image at 24 hours post-injection using an image-enhancing agent with 1 Cy5 dye per polymer (400 polylysine with 96% percentage conjugation of the magnetic resonance imaging moiety). As FIG. 7A shows, the tumor is well highlighted with the low dye load for the image-enhancing agent, and much weaker enhancement is seen using the high dye loaded image-enhancing agent. Of particular note is the lack of definition of the tumor rim in the high load variant.

### Example 12

Signal Change over Time. The question of imaging efficacy can also be addressed in terms of MR signal change with time shortly after agent injection. In the absence of significant blood clearance over the time of signal slope measurement, the rate of change of tumor signal change is proportional to the tumor endothelial permeability for the agent. Endothelial permeability is a hemodynamic parameter of great interest in the staging of tumors. Therefore, agents that give strong uptake give a large signal by which to gauge the tumor permeability, whereas agents that are unable to cross the endothelium at a sufficient rate give a small signal and are not as effective for delineating tumor stage.

In FIG. 8, signal slope for image-enhancing agents of various degrees of percent conjugation of the optical imaging moiety. For Gd-PL (400-mer, 96% conjugation of the magnetic resonance imaging moiety) polymers without any dye attachment, one expects signal slopes in the range of 200% per hour. As the graph in FIG. 8 shows, the Gd-PL-dye agents give nearly equivalent signal uptake slopes to the parent compound over a broad range of dye percentage conjugations, until the dye loading results in a negative charge at each available residue.

FIG. 8 shows the MRI tumor signal enhancement slope (%/hr) in first 30 minutes post tail vein injection (0.025 mmole Gd/kg; 65 nm polymer/kg as a function of mean number of dye molecules per polymer for Gd-DTPA-PLL-Cy5 (˜96% DTPA conjugation). The signal enhancement slope is proportional to the permeability of the agent through the tumor vasculature. The first and the last data points are for n=3 and n=4, and the error bars are the standard deviation in the group measurements. The remaining data are for individual animal experiments and the error bars are the estimated uncertainty of the slope measurement (for 11 dye data, n=3 also). The maximum number of dye per polymer obtained in the synthesis protocol with a excess of dye molecules was 16 occupying most of the remaining available conjugation sites. The permeability of the maximally loaded polymers is seen to be very low which is also seen in the 24-hour images shown in FIG. 7B.

The invention may be embodied in other specific forms without departing from the spirit or essential characteristics thereof. The foregoing embodiments are thereof to be considered in all respects illustrative rather than limiting on the invention described herein. Scope of the invention is thus indicated by the appended claims rather than by the foregoing description, and all changes that come within the meaning and range of equivalency of the claims are therefore intended to be embraced therein.

We claim:

1. An image-enhancing agent comprising a polyamino acid polymer comprising multiple amino residues; a plurality of magnetic resonance imaging moieties covalently bound to the polyamino acid polymer through an amine group; and at least one optical imaging moiety comprising polymethine cyanine dye covalently bound to the polyamino acid polymer through an amine group, wherein at least 1% of the amino residues of the polyamino acid polymer includes an unreacted amine group.

2. The image enhancing agent of claim 1, wherein the polymethine cyanine dye is selected from Cy5, Cy7, meso-substituted Cy5, and meso-substituted Cy7.

3. The image-enhancing agent of claim 1, wherein the polyamino acid polymer comprises 100 to 1000 lysine residues.

4. The image-enhancing agent of claim 3, wherein polyamino acid polymer comprises 200 to 800 lysine residues.

5. The image-enhancing agent of claim 3, wherein the polyamino acid polymer comprises 350 to 450 lysine residues.

6. The image-enhancing agent of claim 1, wherein the magnetic resonance imaging moiety comprises gadolinium.

7. The image-enhancing agent of claim 1, wherein the percentage conjugation of the magnetic resonance imaging moiety is greater than 80% and less than 99%.

8. The image-enhancing agent of claim 7, wherein the percentage conjugation of the magnetic resonance imaging moiety is greater than 90% and less than 99%.

9. The image-enhancing agent claim 1, wherein the percentage conjugation of the polymethine cyanine dye is 0.25%.

10. The image-enhancing agent claim 1, wherein the percentage conjugation of the polymethine cyanine dye is 0.75%.

11. The image-enhancing agent claim 1, wherein the percentage conjugation of the polymethine cyanine dye is between 1% and 3%.

12. The image-enhancing agent claim 1, wherein the percentage conjugation of the polymethine cyanine dye is greater than 3% and less than 5%.

13. A method of imaging comprising the steps of,
(a) administering the image-enhancing agent of claim 1, to the subject; and
(b) imaging the subject using a magnetic resonance technique, an optical imaging technique, or a combination thereof.

14. The method of claim 13, wherein the imaging step comprises a magnetic resonance technique and an optical imaging technique, wherein both techniques occur in a closed surgical field.

15. The method of claim 13, wherein the imaging step comprises a magnetic resonance technique and an optical imaging technique, wherein both techniques occur in an open surgical field.

16. The method of claim 13, wherein the disease tissue is tumor tissue, and the imaging step comprises visualizing the tumor tissue using optical imaging techniques.

17. The method of claim 13, wherein the imaging step occurs more than 6 hours after administration step.

18. The method of claim 13, wherein the imaging step occurs more than 10 hours after administration step.

19. The method of claim 13, wherein the imaging steps occurs at 12 hours after administration step.

20. The method of claim 13, wherein the optically measured target to background ratio for the disease tissue relative to the non-diseased tissue is greater than 2:1.

21. The method of claim 13, wherein the optically measured target to background ratio for the disease tissue relative to the non-diseased tissue is greater than 4:1.

22. A method for managing disease in a body comprising,
(a) distinguishing disease tissue from non-diseases tissue in the body using the image-enhancing agent of claim 1; and
(b) treating the disease tissue.

23. The method of claim 22, wherein the treating step comprises the step of excising at least the disease tissue.

24. The method of claim 22, wherein the treating step comprises administrating a radiotherapuetic agent or a chemotherapeutic agent to at least the disease tissue identified by the image-enhancing agent.

25. The method of claim 22, wherein the distinguishing step and the treating step both occur in a closed surgical field.

26. The method of claim 22, wherein either the identifying step or treating step occurs in an open surgical field.

27. The method of claim 22, wherein the identifying step includes using a magnetic resonance device in a closed surgical field followed by using an optic device in an open or a closed surgical field.
